(19) United States
(12) Patent Application Publication
(10) Pub. No.: US 2004/0249251 A1
(43) Pub. Date: Dec. 9, 2004

(54) System and method for facilitating health care

(76) Inventor: Francis Olschafskie, Boston, MA (US)

Correspondence Address: Elizabeth P. Morano, Bromberg & Sunstein LLP, 125 Summer Street, Boston, MA, 02110-1618, US

(21) Appl. No.: 10/863,060

(22) Filed: Jun. 7, 2004

Related U.S. Application Data
(60) Provisional application No. 60/475,950, filed on Jun. 5, 2003.

(51) Int. Cl. A61B 5/00

(52) U.S. Cl. 600/300; 128/920

(57) ABSTRACT

A method and system for facilitating health care and/or user intake procedures includes providing a website over a computer network to one or more users and providing a knowledge base, which may include one or more databases, containing information related to one or more medical conditions and/or one or more pharmaceuticals. The user is screened with respect to the one or more particular medical conditions and the screening comprises asking the user a plurality of questions related to the medical condition via the website and assessing the user's answers to the questions via a rules-based data structure. A report based on the assessment is then produced.

The present application claims priority from U.S. Provisional Application No. 60/475,950, filed Jun. 5, 2003, which is hereby incorporated herein, in its entirety, by reference.

### TECHNICAL FIELD

The present invention relates to a method of facilitating health care and, in particular to a method of providing a website for medical data intake procedure, screening and assisting diagnosis and treatment of a site user.

### BACKGROUND ART

It is known in the prior art that medical personnel, journals, and books comprise a great wealth of information that may be used to diagnosis and treat patients. A medical facility may involve an intake process and a doctor may interview, review, diagnose and treat a patient. However, often a patient has a medical condition that is reoccurring or a medical condition that can be treated without a doctor's intervention. Intake processing and interviewing a patient can be time consuming and costly for an medial facility, and insurer covering the visit.

### SUMMARY OF THE INVENTION

In a first embodiment of the invention a method for facilitating health care and/or facilitating a user intake process includes providing a website over a computer network to one or more users, providing an intake process to obtain information and/or providing a knowledge base containing information related to one or more users, one or more medical conditions and/or one or more pharmaceuticals. Providing a knowledge base may include providing one or more databases containing information related to user personal information (information specific to one or more users) and/or information related to one or more medical conditions and/or pharmaceuticals. The user is screened with respect to user personal information and/or the one or more particular medical conditions, and the screening includes asking the user a plurality of questions related to his or herself and/or the medical condition via the website and assessing the user's answers to the questions via a knowledge base such as one or more data bases and/or a rules-based data structure. A report based on the assessment, based on the assessment and the user personal information and/or based on the assessment and any other information obtained through the intake process is then produced.

In accordance with related embodiments, preparing a report based on the assessment may include preparing a report that includes suggested treatments for the medical condition. Further preparing a report based on the assessment may include preparing a report that includes further questions to ask the user, a clinician (such as a nurse's aid, nurse, or doctor), or health care practitioner. Additionally, preparing a report based on the assessment may include preparing a report that assists in the diagnosis of the medical condition and/or preparing a report that includes suggesting a diagnosis of a medical condition. Preparing a report based on the intake information and assessment may also include preparing a report that suggests (any use of data) a pharmaceutical and dosage to treat the medical condition.

In accordance with additional related embodiments, providing a website over a computer network to one or more users may include providing the website to a pool of users such that each user is screened prior to visiting or communicating with a clinician or health care practitioner. Further, producing a report based on the assessment may include producing a report with user responses, follow-up suggestions and suggested treatments to be presented to the clinician or health care practitioner.

In accordance with further related embodiments, asking the user a plurality of questions may include asking the user questions related to user personal information and/or general medical history, asking the user questions directly concerning the medical condition, and/or asking the user questions regarding medications the user has taken.

In accordance with still further related embodiments, assessing the user's answers may include comparing answers regarding the medications the user is taking and determining the proper medication to prescribe to the user for treatment of the medical condition and/or comparing two or more possible prescription treatments for the user.

In accordance with other related embodiments, producing a report may include producing a user interview summary, producing a user treatment summary, producing a treatment rating summary, producing a prescription rating summary, displaying feedback based on answers provided by the user and the rules-based data structure, producing one or more electronic displays and/or producing a printed report.

In accordance with another embodiment of the invention, a system for facilitating health care includes a standard treatment module for collecting and producing information related to ratings of various treatments for one or more medical conditions and a first question module in communication with the standard treatment module for generating a first set of questions for the user to answer. A second question module for generating a second set of questions based on the user's answers to the first set of questions is in communication with the first question module and a user history module. The user history module is also in communication with the first question module and collects information relating to the medical history of one or more users and/or user personal information. A treatment comparison module for comparing various pharmaceuticals and other treatments which may be used to treat the medical condition is in communication with the standard treatment module and a feedback module for generating user-specific feedback based on all of the answers that the user provides and on the information contained in the various other modules is in communication with the user history module.

In accordance with a related embodiment the system may include a summary module in communication with the user history module for generating a summary of a user session for the user or a physician. In accordance with a further related embodiment, the system may include a medical condition information module for collecting and producing information related to a particular medical condition. In accordance with yet another related embodiment, the system also includes a user specific treatment module for collecting and producing information relating to pharmaceuticals and other treatments with respect to a particular user.

In accordance with a further embodiment of the invention, a computer program product for facilitating health care (and/or facilitating user intake procedures), the computer program product comprising a computer usable medium having computer readable program code, includes program code for providing a website over a computer network to one or more users and program code for providing a knowledge base containing information related to one or more medical conditions. The knowledge base may include one or more databases containing information related to user personal information, one or more medical conditions and/or one or more pharmaceuticals. Program code for screening the user with respect to user personal information and/or the one or more particular medical conditions is also provided wherein the screening includes asking the user a plurality of questions related to the user and/or related to the medical condition via the website and assessing the user's answers to the questions via a rules-based data structure. The computer program product further includes program code for producing a report based on the assessment and/or based on the assessment and the user personal information.

### BRIEF DESCRIPTION OF THE DRAWINGS

The foregoing features of the invention will be more readily understood by reference to the following detailed description, taken with reference to the accompanying drawings, in which:

FIG. 1 is a block diagram illustrating a system for facilitating health care and/or user intake procedures in accordance with one embodiment of the invention;

FIG. 2 is a flow chart illustrating a method for facilitating health care in accordance with another embodiment of the invention;

FIGS. 3(a)-3(c) are illustrations of a graphical interface for displaying and comparing general treatment ratings in accordance with another embodiment of the invention;

FIGS. 4(a)-4(e) are illustrations of a graphical interface for interviewing a user in accordance with a further embodiment of the invention;

FIGS. 5(a) and 5(b) are illustrations of a graphical interface for displaying overall treatment ratings and user personalized treatment ratings in accordance with a further embodiment of the invention;

FIG. 6 is an illustration of a graphical interface for comparing two or more user specific treatments;

FIG. 7 is an illustration of a graphical interface for displaying the results of the comparison of FIG. 6;

FIG. 8 is an illustration of a graphical interface for providing feedback to the user in accordance with a further embodiment of the invention;

FIG. 9 is an illustration of a graphical interface for providing an interview summary in accordance with another embodiment of the invention; and

FIGS. 10(a)-10(c) are illustrations of graphical interfaces for providing a website for facilitating health care in accordance with a further embodiment of the invention.

### DETAILED DESCRIPTION OF SPECIFIC EMBODIMENTS

The present invention comprises a system and method for facilitating health care and intake procedures through an interview protocol and query system applied to rules-based logic. The system and method incorporates a structured iterative query system, rules content database and dynamic rules processing, report generation, and user interface programs that are intended to be widely transparent and accessible to medical professionals medical cost payers, and health care consumers through computer networks, including the Internet, without limitations related to particular operating systems, browsers or servers. The system generates reports which are organized in a hierarchy of importance relative to matter such as demographic data, age group etc., treatment choices, drug dosage, cautions, contra-indications, side effects, safety, and special circumstances.

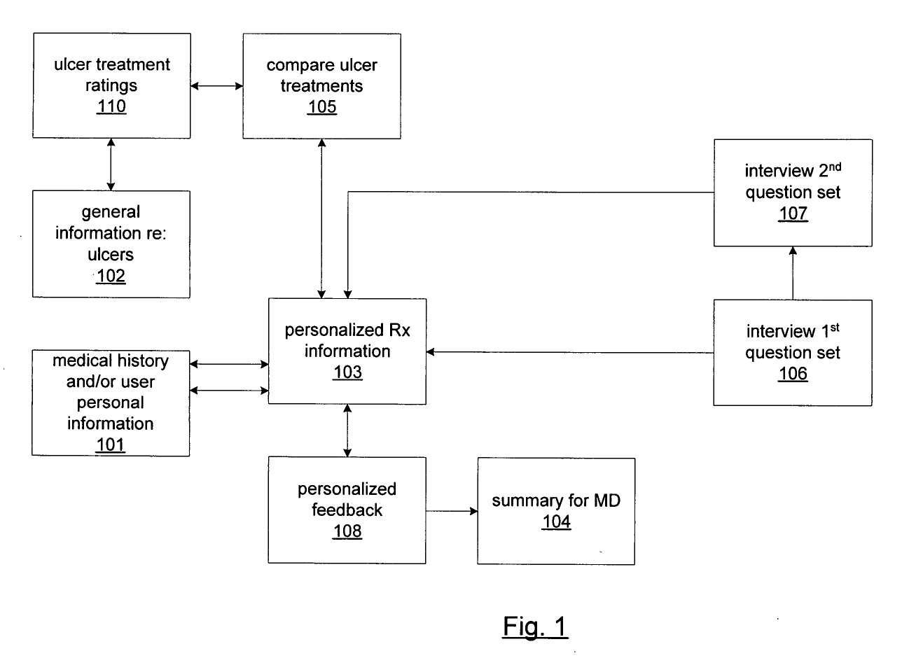

FIG. 1 is a block diagram illustrating a system for facilitating health care in accordance with one embodiment of the invention. The system includes a standard treatment module 110 for collecting and producing information related to ratings of various treatments for a particular medical condition, in this case ulcers. The system also includes a user history module 101 for collecting information relating to the medical history of one or more users and/or user personal information such as user specific physical, emotional and mental attributes and/or habits and/or lifestyle. The system further includes a medical condition information module 102 for collecting and producing information related to standard information on a particular medical condition, a user specific treatment module 103 for collecting and producing information relating to pharmaceuticals and other treatments with respect to a particular user, a summary module 104 for collecting a summary of a user session for the user or a physician and a treatment comparison module 105 for comparing various pharmaceuticals and other treatments which may be used to treat the medical condition. Modules 101, 102, 103, 104, and 105 communicate with module 110 which contains ratings related to various known treatments for the particular condition. A preliminary question module 106 generates a first set of interview questions for the user to answer and follow-up question module 107 generates a follow-up set of questions based on the user's answers to the first set of questions. Feedback module 108 generates user-specific feedback based on all of the answers that the user provides and on the information contained in the various other modules. The modules 101-108 and 110 may be part of a single database resident on a server or computer or they may be resident on one or more servers or computers in communication with one another via a network such as the Internet.

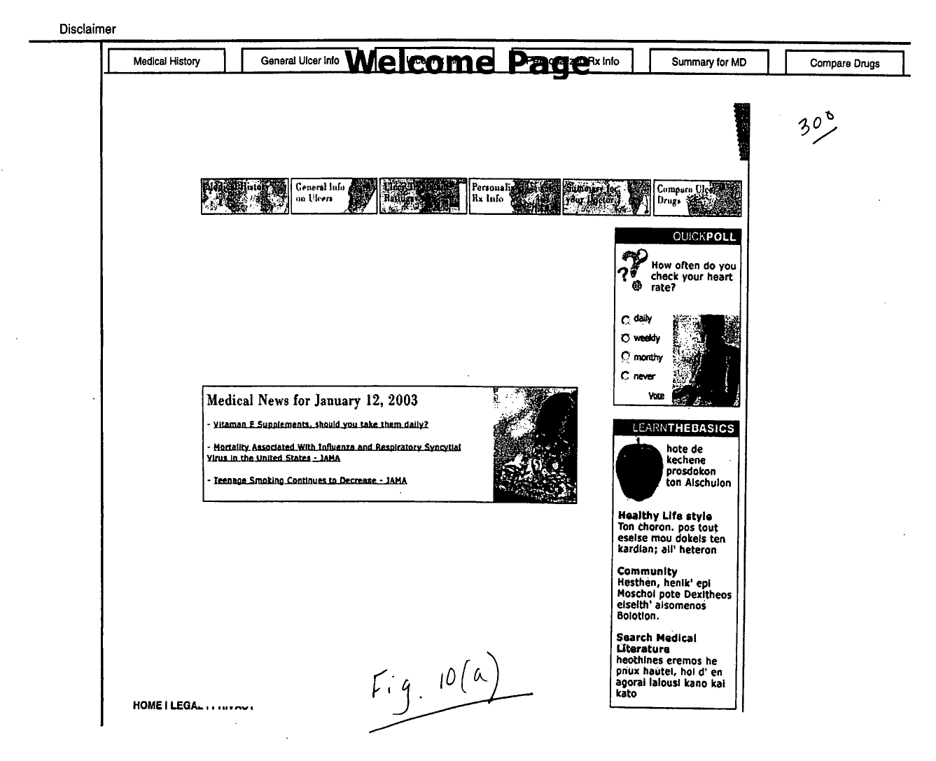

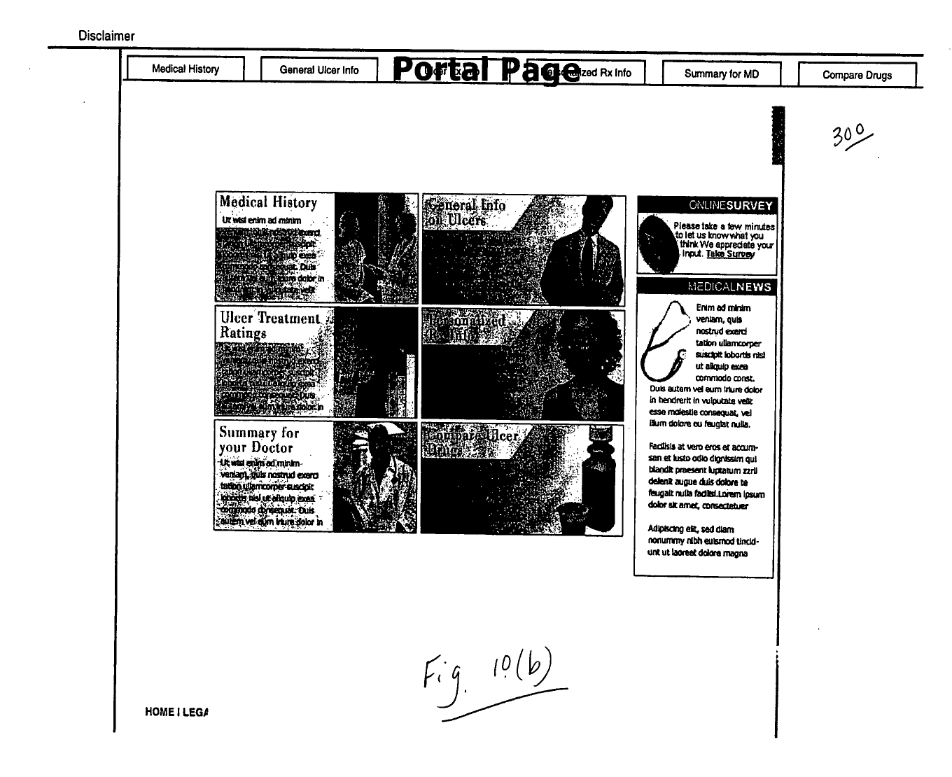

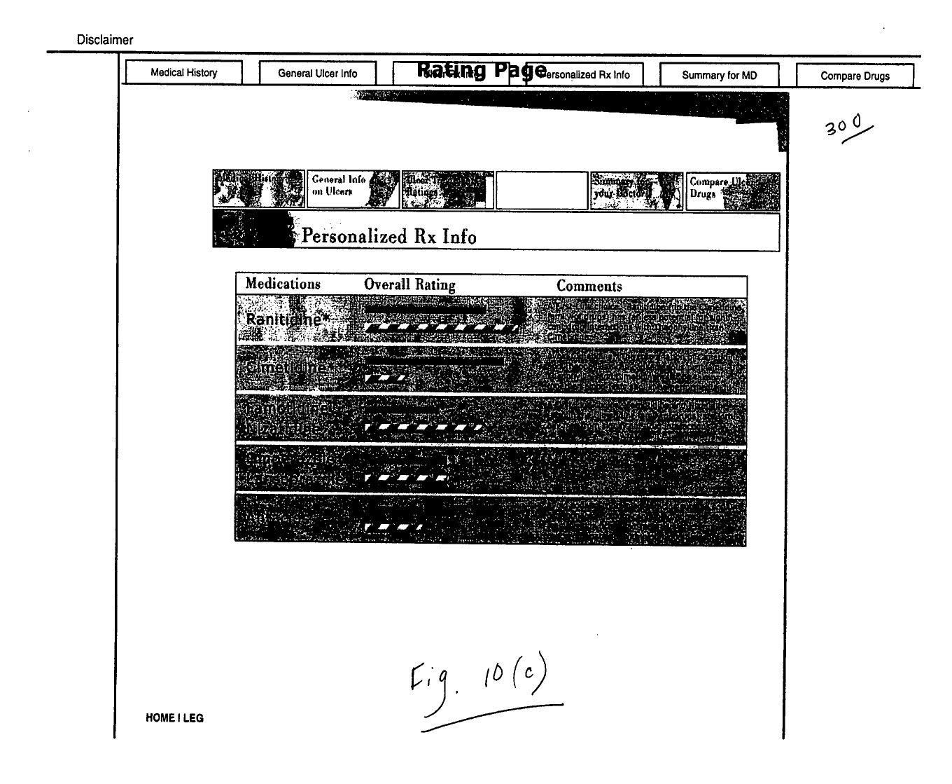

In one embodiment, the system is provided to a user via a website. For example, the system may be provided to one or more subscribers via a health care insurance company's website as shown in FIGS. 10(a)-10(c). For non-emergency situations, the insurance company may mandate that subscribers facilitate medical treatment by consulting the website prior to visiting or communicating with a doctor or other health care practitioner (such as a nurse, nurse's aide or therapist). Following a user session as described in more detail below, the system generates a report that either renders a visit to the health care practitioner or provider unnecessary or facilitates the visit by providing information that is necessary to the proper diagnosis and treatment of the user or patient.

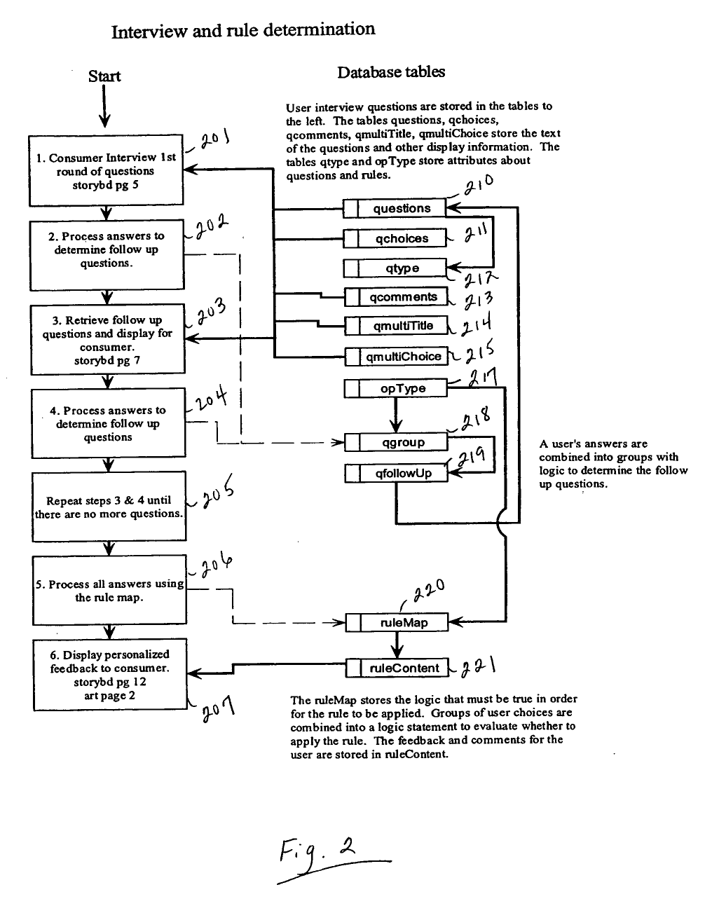

FIG. 2 is a flow chart illustrating a method for facilitating health care and/or user intake procedures in accordance with another embodiment of the invention. In process 201, a first user or consumer interview is conducted. During this process, the user is provided with a first set of questions. The text and display information for these questions are stored in tables 210-215. (In table 217 are stored attributes about the questions and rules.) The user's answers to the first set of questions are assessed 202 to determine what second set or follow-up set of questions, if any, should be presented to the user. The user's answers are combined into groups using rule-based logic and the groups are stored in table 218. The groups are used to determine the second set or follow up questions which are stored in table 219. The follow up questions are retrieved 203 and displayed to the user. The user's answers to the follow up questions are assessed 204 to determine if an additional set of questions should be presented to the user. Processes 203 and 204 are repeated 205 until no further questioning is deemed necessary. All the user's answers are assessed 206 in accordance with a rule map 220. The rule map 220 stores the logic that must be true in order for a rule to be applied. Groups of user choices are combined into a logic statement to evaluate whether to apply a particular rule. Feedback and comments to be displayed to the user are stored in the rule content table 221. Personalized feedback provided by the rule content table is then displayed 207 to the user. Appendix A shows a database schema which may be used to provide the various tables and processes described with respect to the invention.

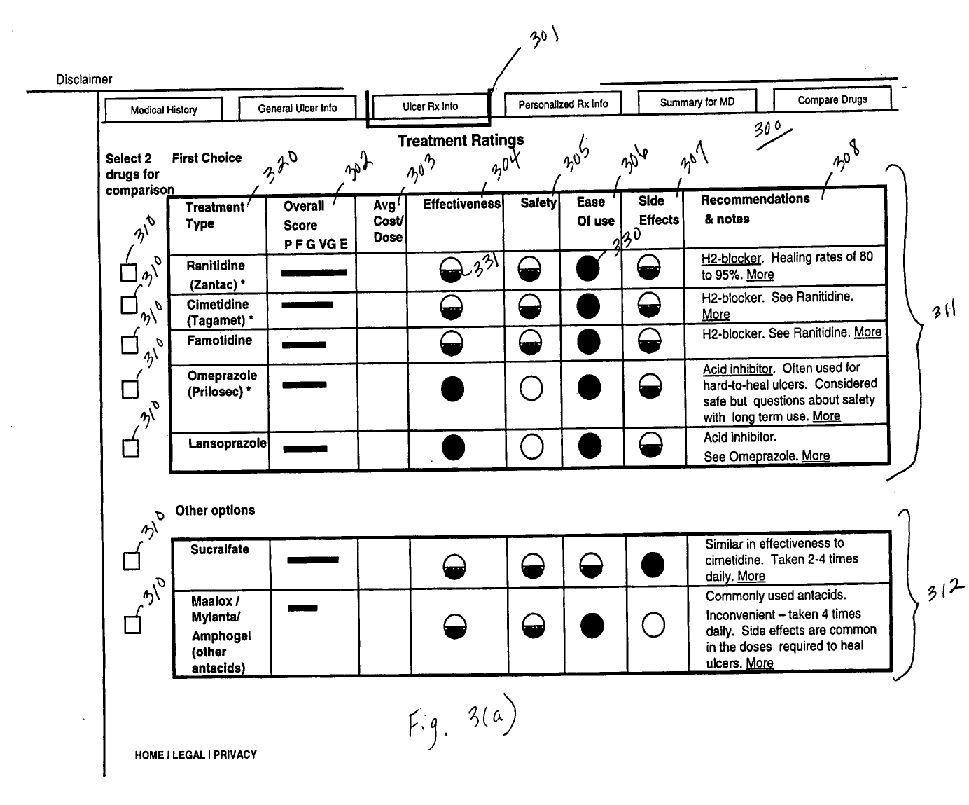

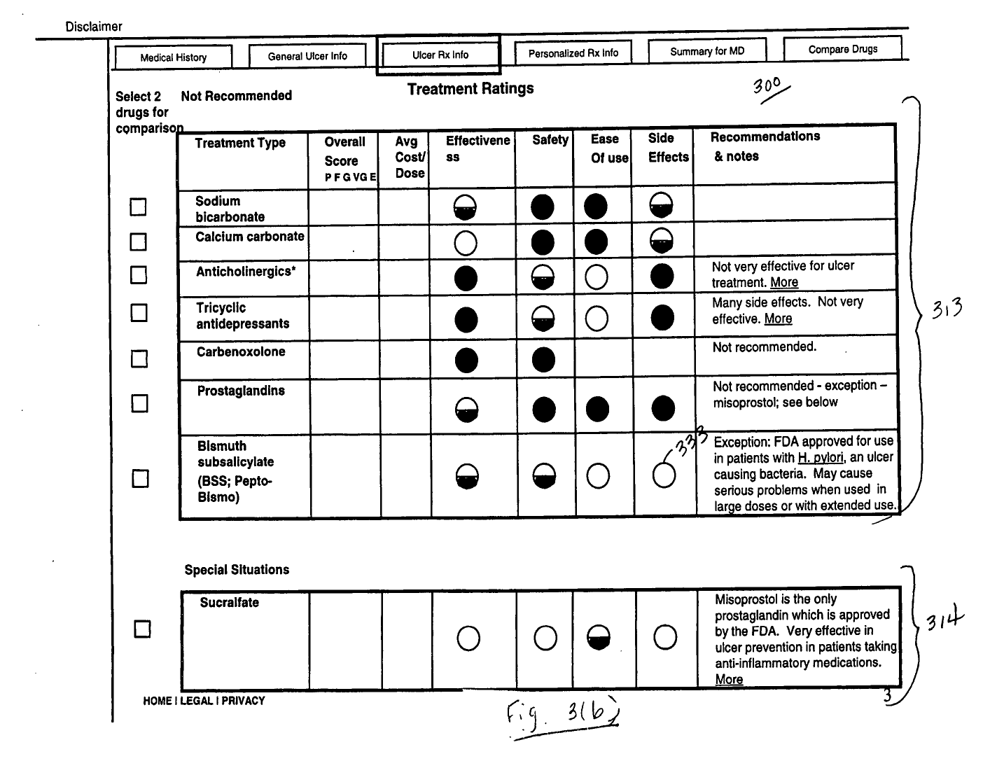

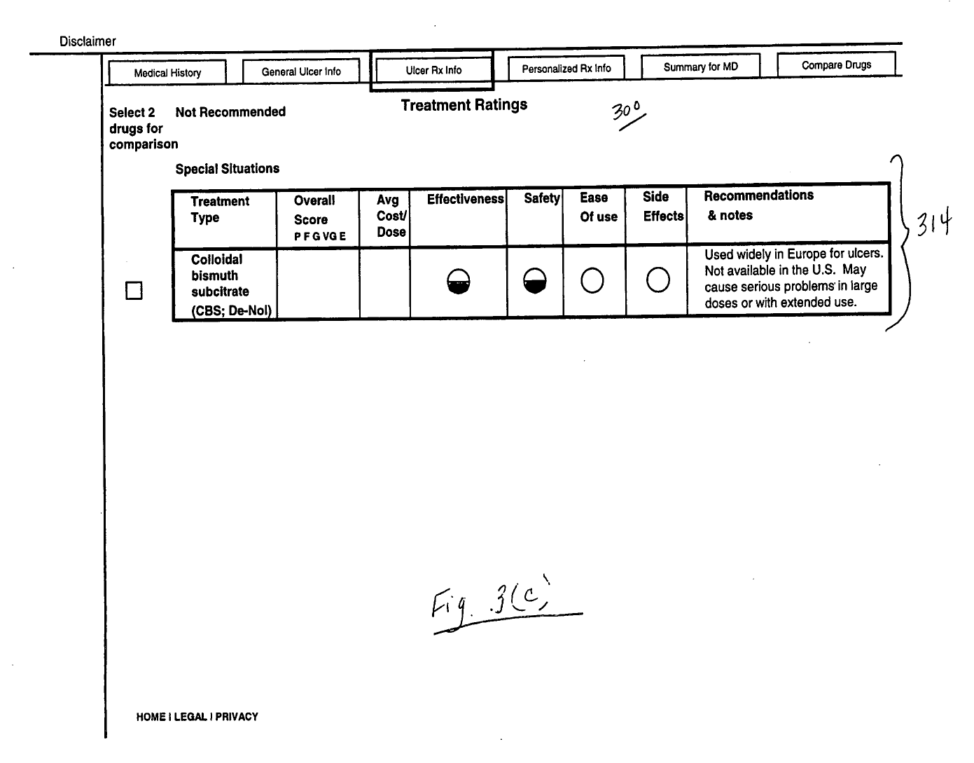

FIGS. 3(a)-3(c) are illustrations of a graphical interface for displaying and comparing general treatment ratings in accordance with another embodiment of the invention. (Note that although particular interfaces are shown in FIGS. 3(a)-10(c), a plurality of different graphical interfaces may be provided to accomplish various tasks associated with the system and methods described herein. Further, graphical user interfaces may be employed to accomplish various other tasks which may require the transfer of information to and from a user (either patient or health care practitioner.) Via a website 300, a user may select tab 301 and standard treatment ratings for a plurality of treatment types 320 for a particular medical condition are displayed in terms of overall score 302, average cost per dose 303, effectiveness 304, safety 305, ease of use 306, and side effects 307. A shaded circle, or some other symbol, appears under each category 302-307 to display the rating for the particular treatment. (A key for the symbols may be displayed in a pull down menu.) For example, half-shaded circle 331 indicates that a particular pharmaceutical, such as Ranitidine®, has been given an effectiveness rating of “very good.” Fully shaded circle 330 indicates that the same treatment has been given a rating of “poor” in terms of ease of use. Un-shaded circle 333 in FIG. 3(b) indicates that another pharmaceutical, such as Bismuth®, has been given a rating of “good” in terms of side effects, and so on.

Recommendations and notes 308 concerning the various treatments are also displayed. The user may check one or more boxes 310 in order to see a display which compares the ratings of two or more of the treatments. As shown in FIG. 3(a), a first choice 311 of treatments are displayed first, other options 312 for treatment are displayed next, and then those treatments which are not recommended 313 are displayed (see FIG. 3(b)). Treatments which may be considered under in accordance with special circumstances 314 are then displayed as shown in FIGS. 3(b) and 3(c).

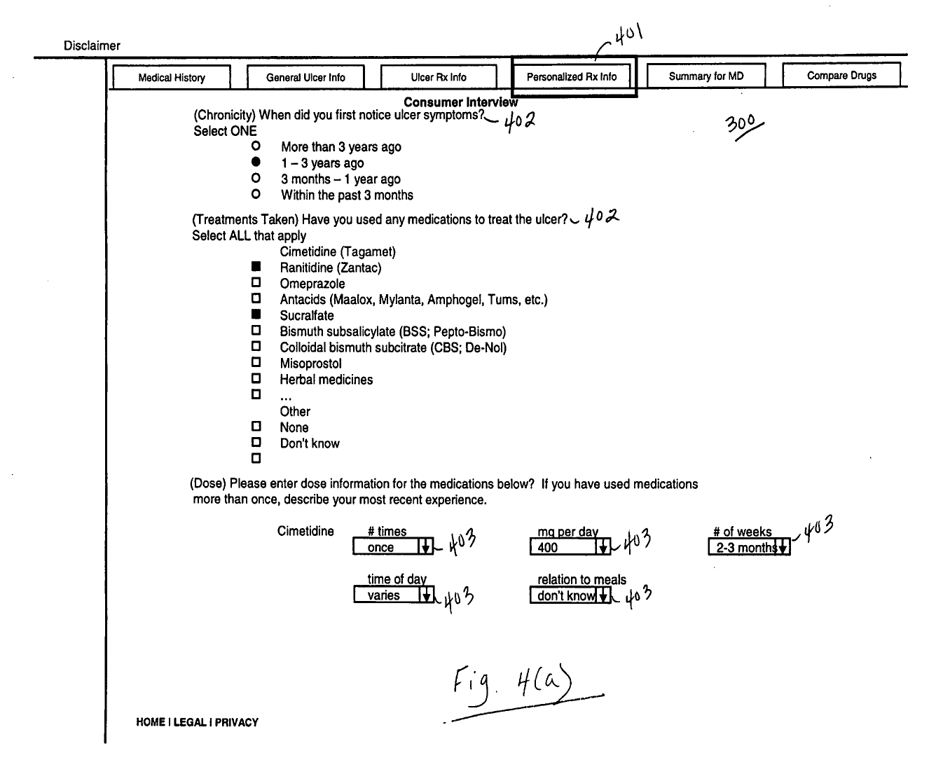

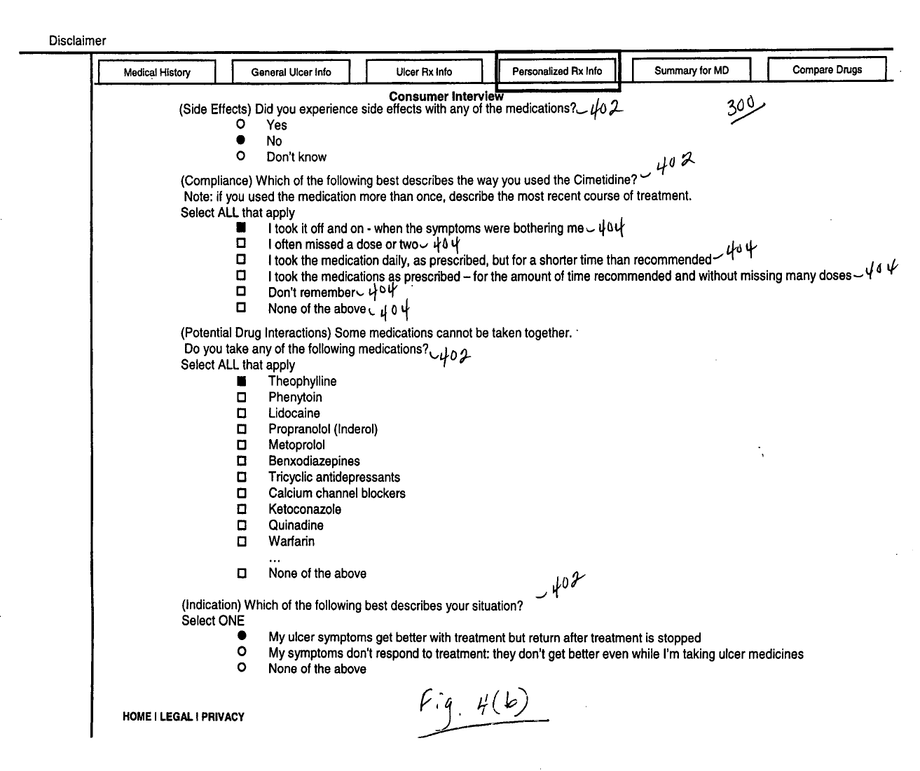

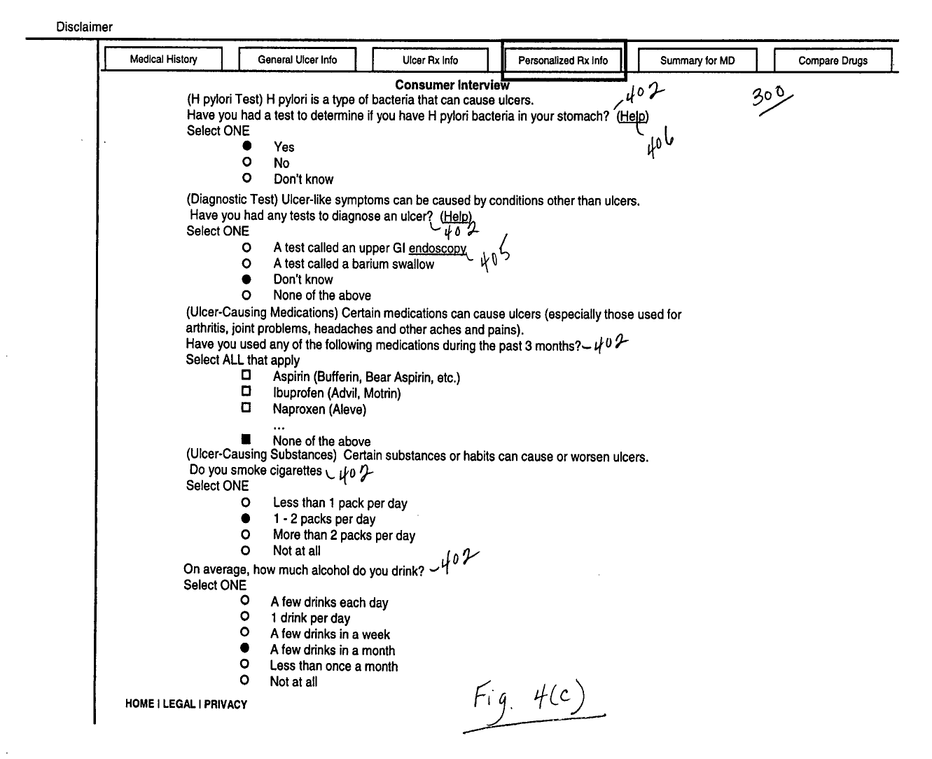

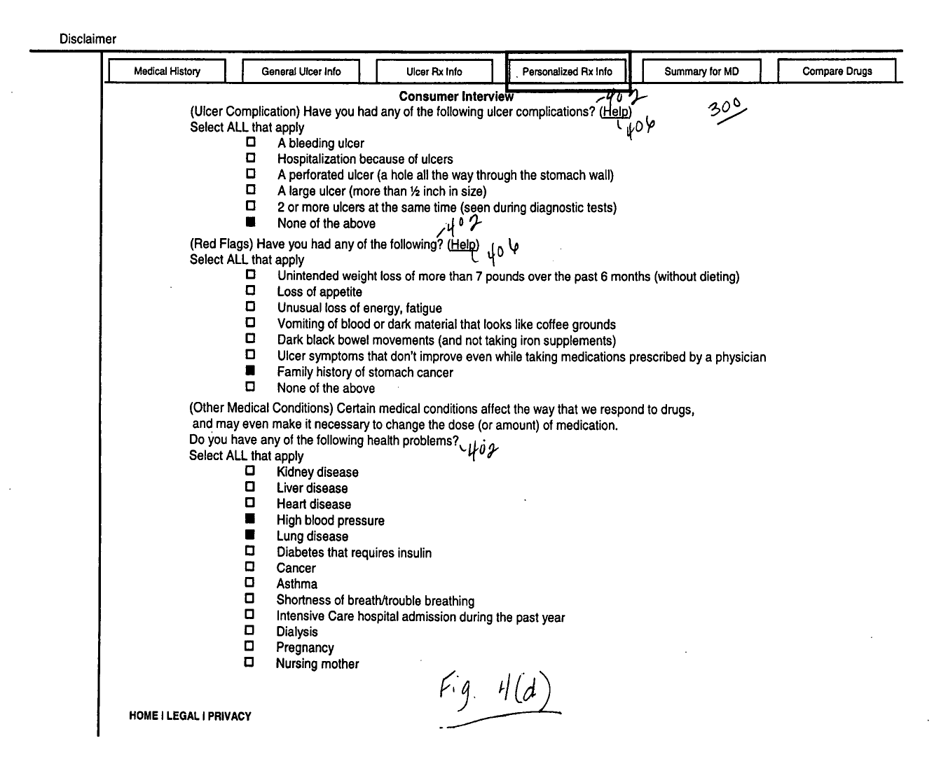

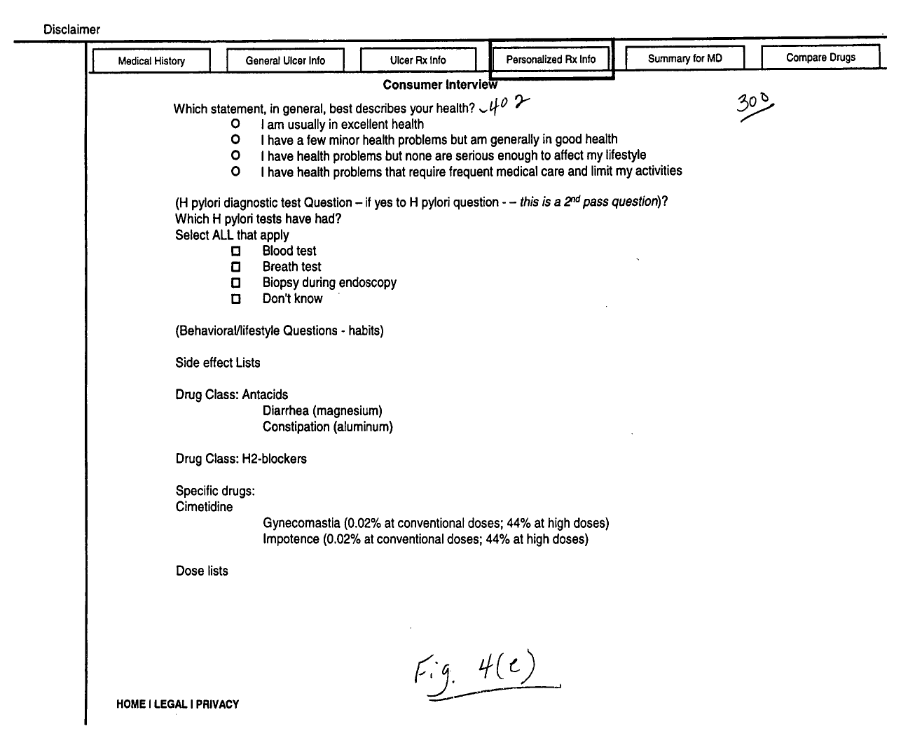

FIGS. 4(a)-4(e) are illustrations of a graphical interface for interviewing a user in accordance with a further embodiment of the invention. By choosing the tab 401 of the website 300, the user may provide user-specific information (or user personal information) by answering questions 402. The user may choose one or more responses 404 from a list of responses for each question. Fields 403 are also provided to enable the user to select from a plurality of answers contained in a pull down menu or to type in an answer that is not provided by the pull down menu. Links 405 and 406 shown in FIGS. 4(c) and 4(d) provide further information and explanations of terminology, such as a glossary of terms, to help the user answer the questions more accurately.

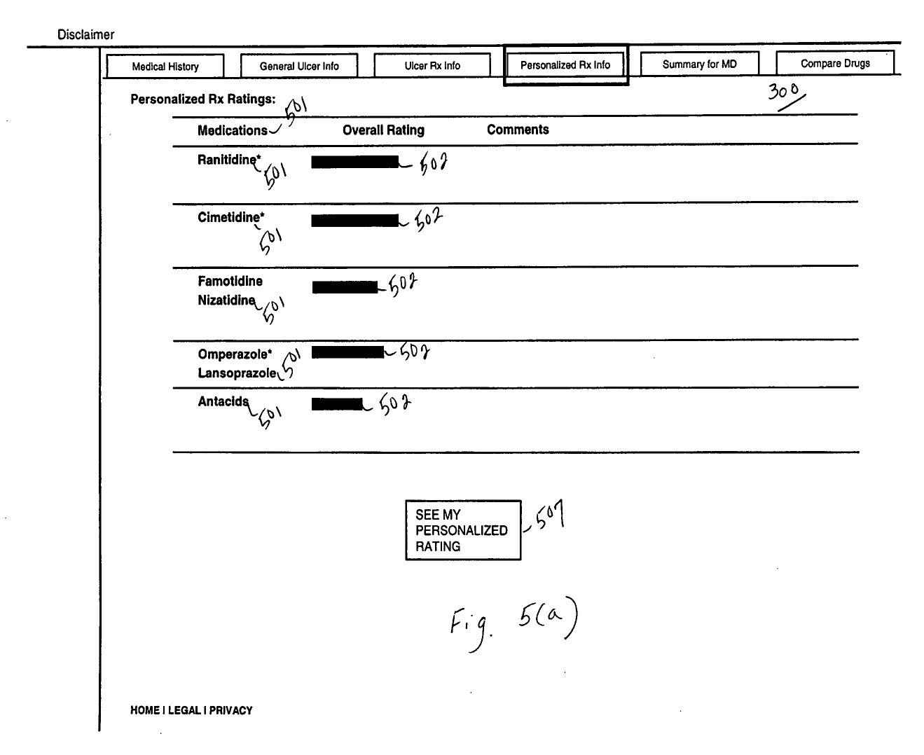

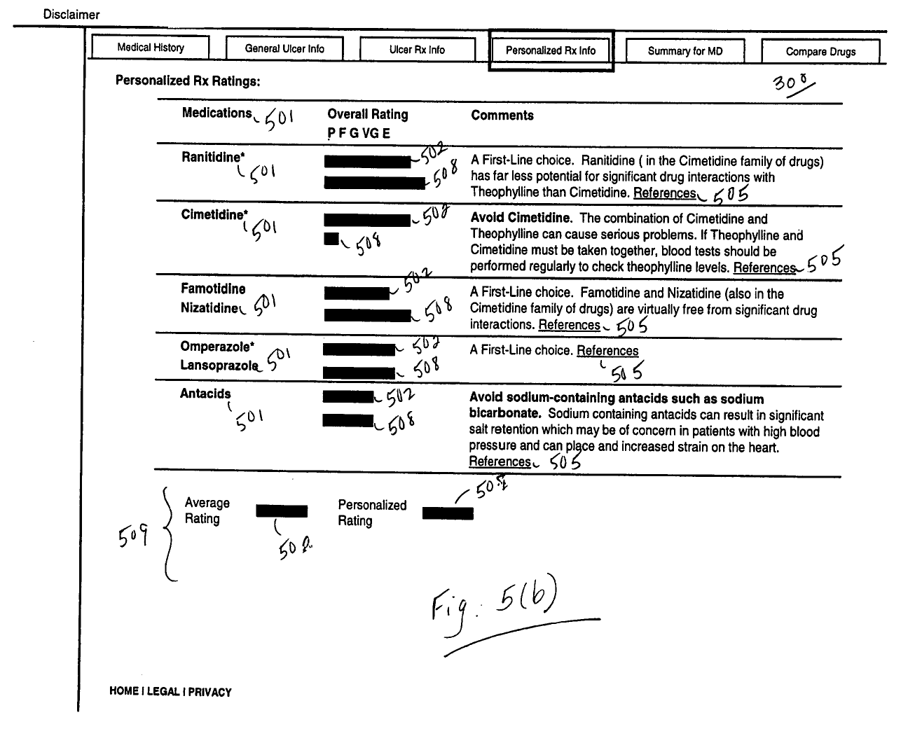

FIGS. 5(a) and 5(b) are illustrations of a graphical interface for displaying overall treatment ratings and user specific treatment ratings in accordance with a further embodiment of the invention. The interface displays a plurality of treatments 501 and the standard overall rating 502 for that treatment. By clicking link 507, a user may see a comparison of the standard overall rating 502 and a user-specific or personalized rating 508 for that user. A key 509 is provided to clarify the meaning the symbols, which in this case are different colored bar graphs. Links 505 provide reference information for each of the treatments.

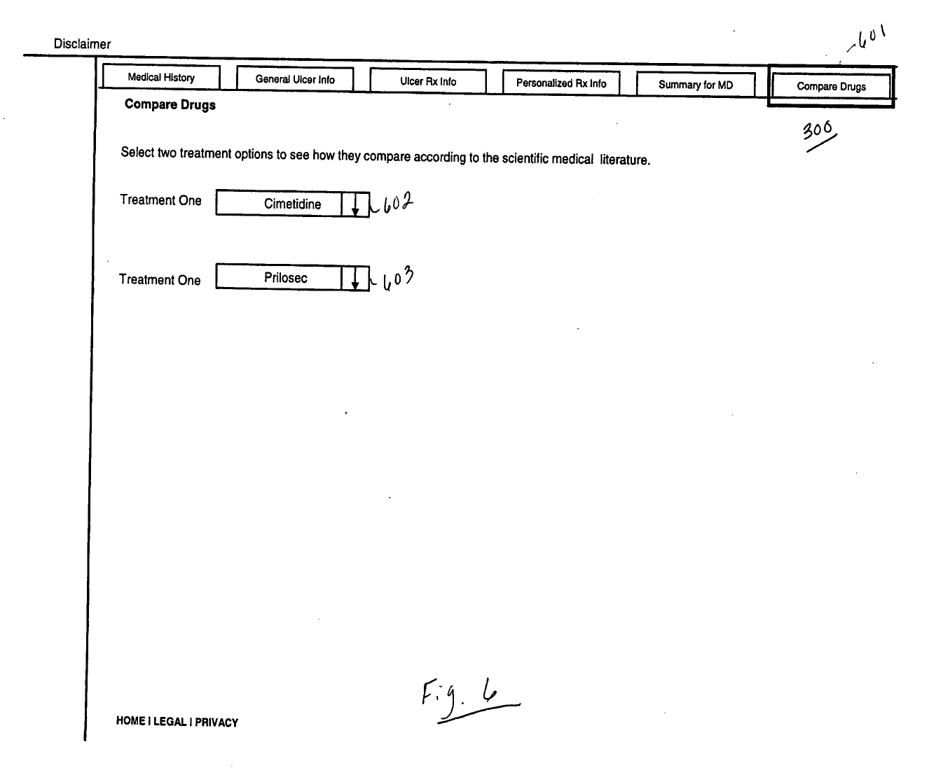

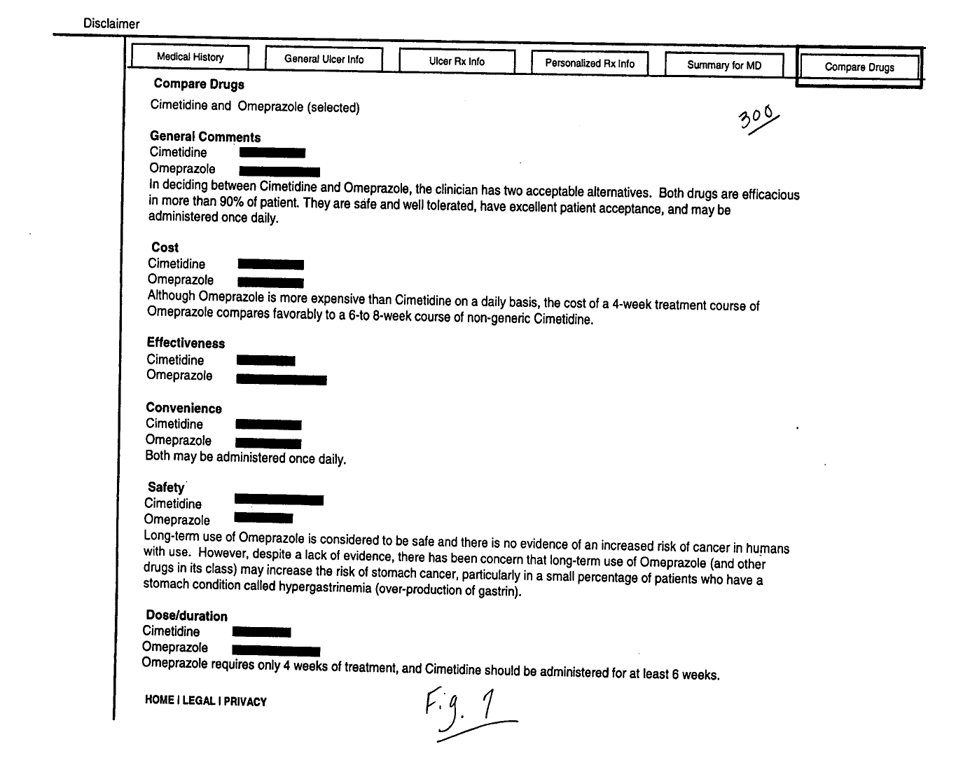

FIG. 6 is an illustration of a graphical interface for comparing two or more user specific treatments. By choosing tab 601 on the website, a user may compare two treatments to one another. This interface provides fields 602 and 603 each of which includes a pull down menu listing a plurality of treatments or pharmaceuticals. A user may choose a treatment from each of the pull down menus and the system will compare the treatments and display the result of the comparison as shown in FIG. 7.

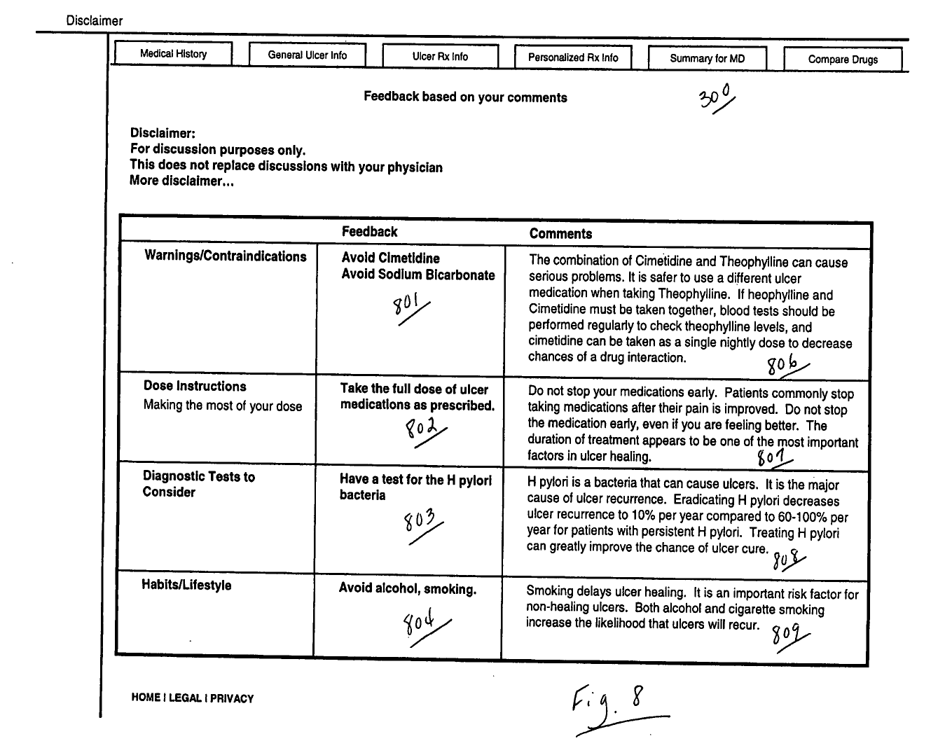

FIG. 8 is an illustration of a graphical interface for providing feedback to the user in accordance with a further embodiment of the invention. This interface provides the user with feedback based on the answers the user supplied and the treatment suggested or selected. The feedback includes including warnings and contraindications 801 regarding a particular treatment, dose instructions 802, diagnostic tests 803 the user should consider, and suggestions regarding the user's habits and lifestyle 804. Comments 806-809 explaining the feedback are also provided.

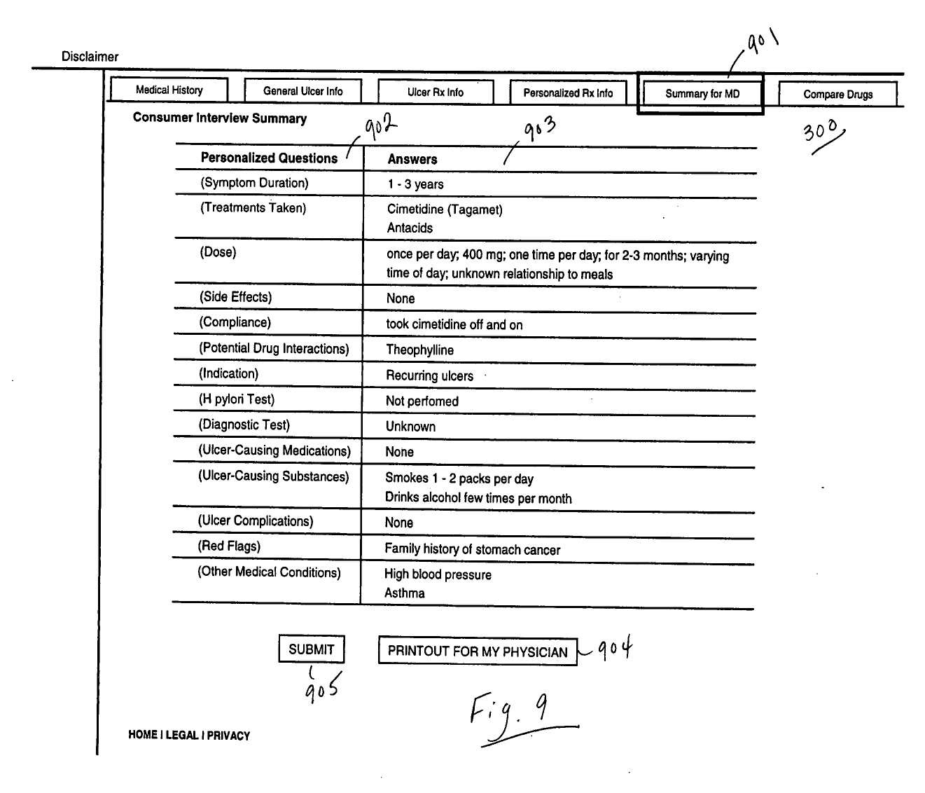

FIG. 9 is an illustration of a graphical interface for providing an interview summary in accordance with another embodiment of the invention. By selecting tab 901 on the website 300, a user may view a summary of his or her session. The subject 902 of questions the user was asked during the interview and the answers 903 that the user supplied are displayed. By clicking hyperlink 905 the summary may be submitted to the system and stored or transmitted to a computer outside of the system. Similarly, by clicking hyperlink 904 a copy of the summary may be printed out. The copy may be presented to the user's physician or health care provider in order to facilitate the diagnosis and treatment of the user.

FIGS. 10(a)-10(c) are illustrations of graphical interfaces for providing a website for facilitating health care and/or facilitating intake procedures in accordance with a further embodiment of the invention. FIG. 10(a) shows a welcome page which may be provided to a pool of users. For example, such a webpage may be provided to people who subscribe to a particular health insurance. The insurance company may provide the website in order to reduce costs associated with office or hospital visits to health care practitioners. For example, the insurance company may mandate that a subscriber be screened through the website prior to going to the doctor or hospital for non-emergency situations. (Similarly, a government agency (e.g., a town, city or federal agency) may provide such a website to a pool of people whose medical costs are paid by the government agency. Likewise, an employer may provide such a website to a pool of employees whose health care costs are paid by the employer.) FIG. 10(b) shows a user portal page by which an insurance subscriber may choose one or more of the modules described with respect to FIG. 1. FIG. 10(b) shows a personalized treatment rating as discussed above.

Although various exemplary embodiments of the invention have been disclosed, it should be apparent to those skilled in the art that various changes and modifications can be made which will achieve some of the advantages of the invention without departing from the true scope of the invention.

What is claimed is:

1. A method for providing enhanced medical decisions comprising:
providing a website over a computer network to one or more users;
providing a knowledge base containing information related to one or more medical conditions;
screening the user with respect to the one or more particular medical conditions, such screening including:
asking the user a plurality of questions related to the medical condition via the website and assessing the user's answers to the questions via a rules-based data structure; and
producing a report based on the assessment.

2. A method according to claim 1, wherein providing a knowledge base includes providing at least one database containing information related to one or more medical conditions.

3. A method according to claim 1, wherein preparing a report based on the assessment includes preparing a report that includes suggested treatments for the medical condition.

4. A method according to claim 1, wherein preparing a report based on the assessment includes preparing a report that includes further questions to ask the user.

5. A method according to claim 1, wherein preparing a report based on the assessment includes preparing a report that includes further questions to ask a health care practitioner.

6. A method according to claim 1, wherein preparing a report based on the assessment includes preparing a report that includes a suggested diagnosis of the medical condition.

7. A method according to claim 1, wherein preparing a report based on the assessment includes preparing a report that includes a suggested pharmaceutical and dosage to treat the medical condition.

8. A method according to claim 1, wherein providing a website over a computer network to one or more users includes providing the website to a pool of users such that each user is screened prior to visiting a health care practitioner.

9. A method according to claim 1, wherein providing a website over a computer network to one or more users includes providing the website to a pool of users such that each user is screened prior to communicating with a health care practitioner.

10. A method according to either of claims 8 or 9, wherein producing a report based on the assessment includes producing a report with user responses, follow-up suggestions and suggested treatments to be presented to the health care practitioner.

11. A method according to claim 1, wherein asking the user a plurality of questions includes asking the user questions related to the user's general medical history.

12. A method according to claim 1, wherein asking the user a plurality of questions includes asking the user questions directly concerning the medical condition.

13. A method according to claim 1, wherein asking the user a plurality of questions includes asking the user questions regarding medications the user has taken.

14. A method according to claim 13, wherein assessing the user's answers includes comparing answers regarding the medications the user is taking and determining the proper medication to prescribe to the user for treatment of the medical condition.

15. A method according to claim 1, wherein assessing the user's answers includes comparing two or more possible prescription treatments for the user.

16. A method according to claim 1, wherein producing a report includes producing a user interview summary.

17. A method according to claim 1, wherein producing a report includes producing user treatment summary.

18. A method according to claim 1, wherein producing a report includes producing a treatment rating summary.

19. A method according to claim 1, wherein producing a report includes producing a prescription rating summary.

20. A method according to claim 1, where producing a report includes displaying feedback based on answers provided by the user and the rules-based data structure.

21. A method according to claim 1, wherein producing a report includes producing one or more electronic displays.

22. A method according to claim 1, wherein producing a report includes producing a printed report.

23. A system for providing enhanced medical decisions comprising:
a standard treatment module for collecting and producing information related to ratings of various treatments for one or more medical conditions;
a first question module in communication with the standard treatment module for generating a first set of questions for the user to answer;
a second question module in communication with the first question module for generating a second set of questions based on the user's answers to the first set of questions;
a user history module in communication with the first question module and the second question module for collecting information relating to the medical history of one or more users;
a treatment comparison module in communication with the standard treatment module for comparing various pharmaceuticals and other treatments which may be used to treat the medical condition; and
a feedback module in communication with the user history module for generating user-specific feedback based on all of the answers that the user provides and on the information contained in the other modules.

24. A system according to claim 23, further comprising:
a summary module in communication with the user history module for generating a summary of a user session for a user or a physician.

25. A system according to claim 23, further comprising:
a medical condition information module for collecting and producing information related to a particular medical condition.

26. A system according to claim 23, further comprising:
a user specific treatment module for collecting and producing information relating to pharmaceuticals and other treatments with respect to a particular user.

27. A computer program product for providing enhanced medical decisions, the computer program product comprising a computer usable medium having computer readable program code including:
program code for providing a website over a computer network to one or more users;
program code for providing a knowledge base containing information related to one or more medical conditions;
program code for screening the user with respect to the one or more particular medical conditions, such screening including:
asking the user a plurality of questions related to the medical condition via the website and assessing the user's answers to the questions via a rules-based data structure; and
program code for producing a report based on the assessment.

28. A computer program product according to claim 27, wherein the knowledge base includes at least one database.

29. A computer program product according to claim 27, further comprising program code for providing a knowledge base containing information related to one or more pharmaceuticals.

30. A computer program product according to claim 27, wherein the knowledge base includes at least one database.
